(19) United States
(12) Patent Application Publication
(10) Pub. No.: US 2022/0028546 A1
(43) Pub. Date: Jan. 27, 2022

(54) ASSESSING THE GAIT OF PARKINSON'S PATIENTS

(71) Applicant: INTERNATIONAL BUSINESS MACHINES CORPORATION, ARMONK, NY (US)

(72) Inventor: Erhan Bilal, Westport, CT (US)

(21) Appl. No.: 16/938,084

(22) Filed: Jul. 24, 2020

(51) Int. Cl. G16H 50/20; G06N 3/04; G06N 3/08; G16H 50/30; G16H 20/10; G16H 50/50; G16H 10/60; A61B 5/11; A61B 5/00

(52) U.S. Cl. CPC G16H 50/20; G16H 40/67; G06N 3/088; G16H 50/30; G16H 20/10; G16H 50/50; G16H 10/60; A61B 5/112; A61B 5/7267; A61B 5/1116; A61B 5/1101; A61B 5/1121; A61B 5/4519; A61B 5/4082; G06N 3/0454

(57) ABSTRACT

The exemplary embodiments disclose a system and method, a computer program product, and a computer system for assessing a user's gate. The exemplary embodiments may include collecting data corresponding to a walking user and assessing a gait of the user based on applying one or more models to the data.

### BACKGROUND

The exemplary embodiments relate generally to assessing user mobility, and more particularly to assessing the gait of patients with Parkinson's disease by collecting gait data.

Gait refers to the manner in which a person walks and can provide insight into the health conditions of a person. For example, gait correlates highly with various diseases, including Parkinson's disease. Parkinson's disease is a neurological condition that affects millions of individuals worldwide with symptoms of tremors, slowness of movement, muscular rigidity, and gait and balance disturbances, among others. The Unified Parkinson's Disease Rating Scale (UPDRS) includes a Postural Instability and Gait Disturbance (PIGD) test, which is often used by medical professionals to score a patient's gait and postural deficits on a scale of 0 to 4. The PIGD test is highly subjective and has poor correlation and consistency between different medical professionals. It can therefore be very difficult for medical professionals to objectively assess the gait of patients with Parkinson's disease.

### SUMMARY

The exemplary embodiments disclose a system and method, a computer program product, and a computer system for assessing a user's gate. The exemplary embodiments may include collecting data corresponding to a walking user and assessing a gait of the user based on applying one or more models to the data.

### BRIEF DESCRIPTION OF THE SEVERAL VIEWS OF THE DRAWINGS

The following detailed description, given by way of example and not intended to limit the exemplary embodiments solely thereto, will best be appreciated in conjunction with the accompanying drawings, in which:

FIG. 1 depicts an exemplary schematic diagram of a gait assessor system 100, in accordance with the exemplary embodiments.

FIG. 2 depicts an exemplary flowchart illustrating the operations of a gait assessor 134 of the gait assessor system 100 in assessing the gait of a user, in accordance with the exemplary embodiments.

FIG. 3 depicts an exemplary schematic diagram of an inertial sensor 124 of the gait assessor system 100 positioned on a user's lower back, in accordance with the exemplary embodiments.

FIG. 4-5 depict exemplary flowcharts illustrating the operations of the gait assessor 134 of the gait assessor system 100 in training a neural network based on training data, in accordance with the exemplary embodiments.

FIG. 6 depicts a graph of results of the gait assessor 134 trained according to the method depicted in FIG. 4-5, in accordance with the exemplary embodiments.

FIG. 7 depicts an exemplary block diagram depicting the hardware components of the gait assessor system 100 of FIG. 1, in accordance with the exemplary embodiments.

FIG. 8 depicts a cloud computing environment, in accordance with the exemplary embodiments.

FIG. 9 depicts abstraction model layers, in accordance with the exemplary embodiments.

The drawings are not necessarily to scale. The drawings are merely schematic representations, not intended to portray specific parameters of the exemplary embodiments. The drawings are intended to depict only typical exemplary embodiments. In the drawings, like numbering represents like elements.

### DETAILED DESCRIPTION OF THE SEVERAL VIEWS OF THE DRAWINGS

Detailed embodiments of the claimed structures and methods are disclosed herein; however, it can be understood that the disclosed embodiments are merely illustrative of the claimed structures and methods that may be embodied in various forms. The exemplary embodiments are only illustrative and may, however, be embodied in many different forms and should not be construed as limited to the exemplary embodiments set forth herein. Rather, these exemplary embodiments are provided so that this disclosure will be thorough and complete, and will fully convey the scope to be covered by the exemplary embodiments to those skilled in the art. In the description, details of well-known features and techniques may be omitted to avoid unnecessarily obscuring the presented embodiments.

References in the specification to “one embodiment”, “an embodiment”, “an exemplary embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to implement such feature, structure, or characteristic in connection with other embodiments whether or not explicitly described.

In the interest of not obscuring the presentation of the exemplary embodiments, in the following detailed description, some processing steps or operations that are known in the art may have been combined together for presentation and for illustration purposes and in some instances may have not been described in detail. In other instances, some processing steps or operations that are known in the art may not be described at all. It should be understood that the following description is focused on the distinctive features or elements according to the various exemplary embodiments.

Gait refers to the manner in which a person walks and can provide insight into the health conditions of a person. For example, gait correlates highly with various diseases, including Parkinson's disease. Parkinson's disease is a neurological condition that affects millions of individuals worldwide with symptoms of tremors, slowness of movement, muscular rigidity, and gait and balance disturbances, among others. The Unified Parkinson's Disease Rating Scale (UPDRS) includes a Postural Instability and Gait Disturbance (PIGD) test, which is often used by medical professionals to score a patient's gait and postural deficits on a scale of 0 to 4. The PIGD test is highly subjective and has poor correlation and consistency between different medical professionals. It can therefore be very difficult for medical professionals to objectively assess the gait of patients with Parkinson's disease.

Exemplary embodiments are directed to a method, computer program product, and computer system that will assess the gait of a user. In embodiments, machine learning may be used to create models capable of assessing gait or postural deficiencies, while feedback loops may improve upon such models. Moreover, data from sensors, the internet, social networks, and user profiles may be utilized to collect data for gait and/or postural assessments. The various data may be indicative of tremors, slowness of movement, muscular rigidity, gait speed, stride length, toe off angle, strike angle, trunk coronal range of motion, body symmetry, etc. In general, it will be appreciated that embodiments described herein may relate to aiding in the determination or assessment of the gait of any human within any environment and for any motivation.

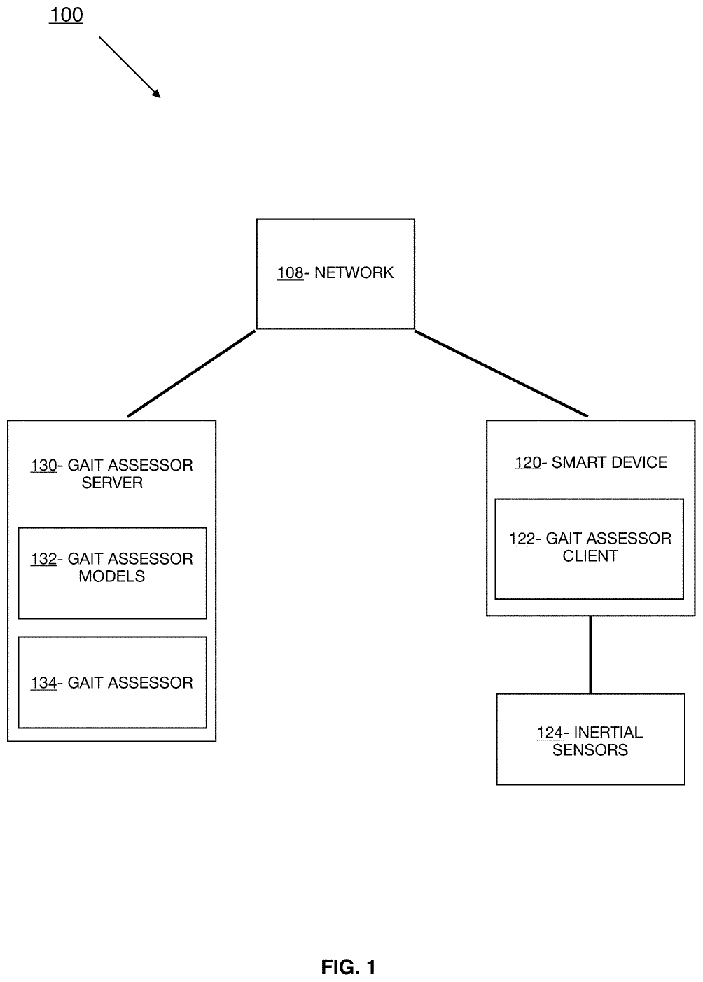

FIG. 1 depicts the gait assessor system 100, in accordance with the exemplary embodiments. According to the exemplary embodiments, the gait assessor system 100 may include a smart device 120 and a gait assessor server 130, which may be interconnected via a network 108. While programming and data of the exemplary embodiments may be stored and accessed remotely across several servers via the network 108, programming and data of the exemplary embodiments may alternatively or additionally be stored locally on as few as one physical computing device or amongst other computing devices than those depicted.

In the exemplary embodiments, the network 108 may be a communication channel capable of transferring data between connected devices. Accordingly, the components of the gait assessor system 100 may represent network components or network devices interconnected via the network 108. In the exemplary embodiments, the network 108 may be the Internet, representing a worldwide collection of networks and gateways to support communications between devices connected to the Internet. Moreover, the network 108 may utilize various types of connections such as wired, wireless, fiber optic, etc. which may be implemented as an intranet network, a local area network (LAN), a wide area network (WAN), or a combination thereof. In further embodiments, the network 108 may be a Bluetooth network, a Wi-Fi network, or a combination thereof. In yet further embodiments, the network 108 may be a telecommunications network used to facilitate telephone calls between two or more parties comprising a landline network, a wireless network, a closed network, a satellite network, or a combination thereof. In general, the network 108 may represent any combination of connections and protocols that will support communications between connected devices.

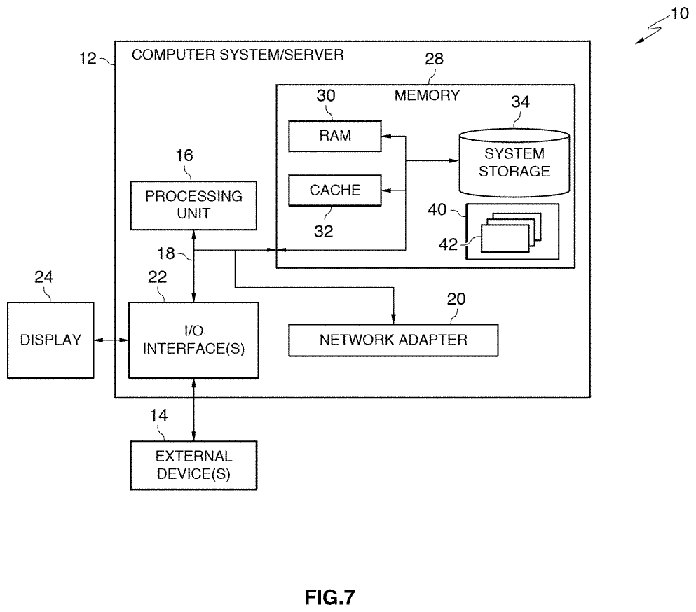

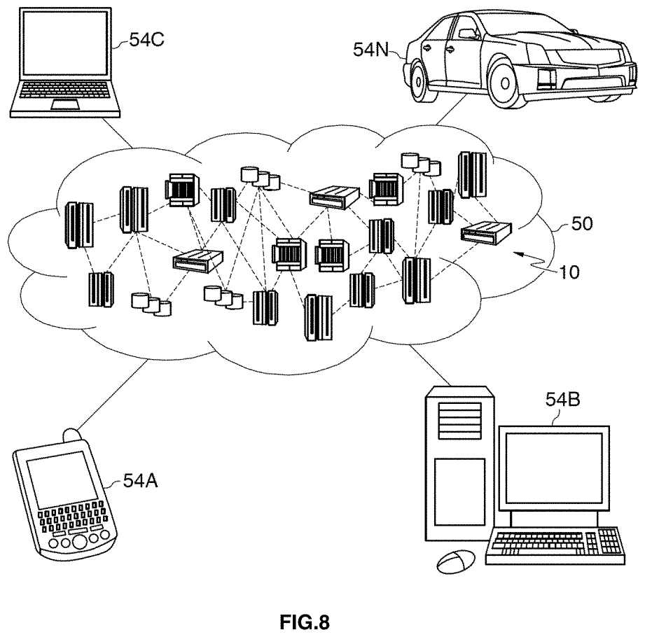

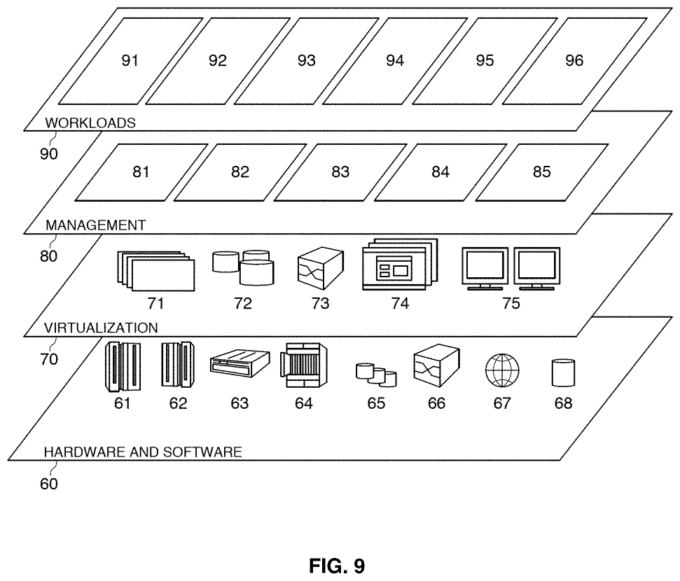

In the example embodiment, the smart device 120 includes a gait assessor client 122 and may be an enterprise server, a laptop computer, a notebook, a tablet computer, a netbook computer, a personal computer (PC), a desktop computer, a server, a personal digital assistant (PDA), a rotary phone, a touchtone phone, a smart phone, a mobile phone, a virtual device, a thin client, an IoT device, or any other electronic device or computing system capable of receiving and sending data to and from other computing devices. While the smart device 120 is shown as a single device, in other embodiments, the smart device 120 may be comprised of a cluster or plurality of computing devices, in a modular manner, etc., working together or working independently. The smart device 120 is described in greater detail as a hardware implementation with reference to FIG. 7, as part of a cloud implementation with reference to FIG. 8, and/or as utilizing functional abstraction layers for processing with reference to FIG. 9.

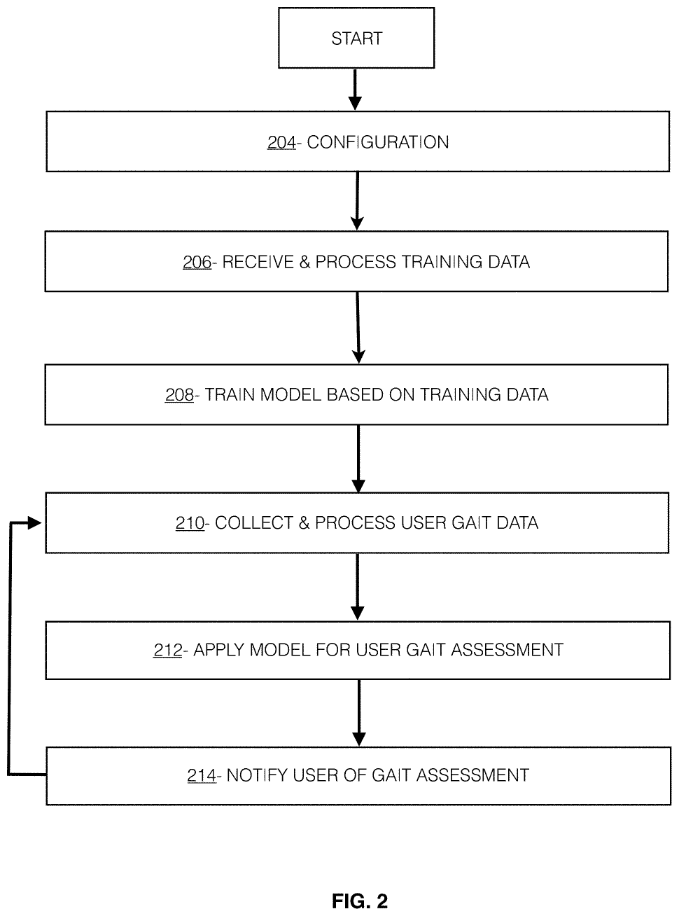

The gait assessor client 122 may be a software and/or hardware application capable of communicating with and providing a user interface for a user to interact with a server via the network 108. The gait assessor client 122 may act as a client in a client-server relationship. Moreover, in the example embodiment, the gait assessor client 122 may be capable of transferring data between the smart device 120 and the gait assessor server 130 via the network 108. In embodiments, the gait assessor 134 utilizes various wired and wireless connection protocols for data transmission and exchange, including Bluetooth, 2.4 gHz and 5 gHz internet, near-field communication, Z-Wave, Zigbee, etc. The gait assessor client 122 is described in greater detail with respect to FIG. 2.

In the exemplary embodiments, the one or more inertial sensors 124 may be an accelerometer, gyroscope, and/or compass, and may additionally include a camera, microphone, light sensor, infrared sensor, movement detection sensor, pressure sensor, or other sensory hardware/software equipment. In embodiments, the inertial sensors 124 may be integrated with and communicate directly with the smart device 120, e.g., smart phones and laptops. Although the inertial sensors 124 are depicted as external to the smart device 120, in embodiments, the inertial sensors 124 may be integrated within smart device 120 or connected to the smart device 120 or the network 108. In embodiments, the inertial sensors 124 may be incorporated within an environment in which the gait assessor system 100 is implemented. For example, the inertial sensors 124 may include a series of video cameras fastened to the walls of a medical facility. The inertial sensors 124 are described in greater detail with respect to FIG. 2 and FIG. 7-9.

In the exemplary embodiments, the gait assessor server 130 includes one or more gait assessor models 132 and a gait assessor 134. The gait assessor server 130 may act as a server in a client-server relationship with the gait assessor client 122, and may be an enterprise server, a laptop computer, a notebook, a tablet computer, a netbook computer, a PC, a desktop computer, a server, a PDA, a rotary phone, a touchtone phone, a smart phone, a mobile phone, a virtual device, a thin client, an IoT device, or any other electronic device or computing system capable of receiving and sending data to and from other computing devices. While the gait assessor server 130 is shown as a single device, in other embodiments, the gait assessor server 130 may be comprised of a cluster or plurality of computing devices, working together or working independently. The gait assessor server 130 is described in greater detail as a hardware implementation with reference to FIG. 7, as part of a cloud implementation with reference to FIG. 8, and/or as utilizing functional abstraction layers for processing with reference to FIG. 9.

The gait assessor models 132 may be one or more algorithms modelling a correlation between one or more features and a gait assessment. The one or more features may include characteristics relating to an user's gait, such as tremors, slowness of movement, muscular rigidity, gait speed, stride length, toe off angle, strike angle, trunk coronal range of motion, body symmetry, etc., and may be detected and extracted via the one or more inertial sensors 124 and the network 108. In embodiments, the gait assessor models 132 may weight the features based on an effect that the one or more features have on the assessment of gait. In the example embodiment, the gait assessor 134 may generate the gait assessor models 132 using machine learning methods, such as neural networks, deep learning, hierarchical learning, Gaussian Mixture modelling, Hidden Markov modelling, and K-Means, K-Medoids, or Fuzzy C-Means learning, etc. The gait assessor models 132 are described in greater detail with reference to FIG. 2.

The gait assessor 134 may be a software and/or hardware program capable of receiving a configuration of the gait assessor system 100. In addition, the gait assessor 134 may be further configured for receiving and processing training data, as well as training one or more gait assessor models 134 and/or neural networks based on the training data. Moreover, the gait assessor 134 may be further configured for collecting and processing a user's gait data, as well as applying the one or more gait assessor models 134 and/or neural networks to assess the user's gait. The gait assessor 134 is described in greater detail with reference to FIG. 2.

FIG. 2 depicts an exemplary flowchart illustrating the operations of a gait assessor 134 of the gait assessor system 100 in assessing a user's gait, in accordance with the exemplary embodiments.

The gait assessor 134 may receive a configuration (step 204). The gait assessor 134 may be configured by receiving information such as a user registration. The user registration may be uploaded by a user, i.e., the owner of the gait assessor system 100, user of the gait assessor system 100, a medical professional who oversees the usage of the gait assessor system 100, a guardian of a minor who uses the gait assessor system 100, an employer who oversees the usage of the gait assessor system 100, etc. In the example embodiment, the user refers to the person for whom gait is being assessed, and the configuration may be received by the gait assessor 134 via the gait assessor client 122 and the network 108. Receiving the user registration may involve referencing a user profile via user login credentials, internet protocol (IP) address, media access control (MAC) address, etc., or receiving user input information such as a name, date of birth, gender, address/geographic information, phone number, email address, company name, device serial number, smart device 120 type, and the like. Receiving a user registration may also involve receiving health data via user input or reference to an electronic medical/health record that includes data relevant to general user health, medical conditions, medications prescribed to the user, information about past medical office visits, information about primary care physicians, etc. Lastly, the gait assessor 134 may receive a configuration of the one or more inertial sensors 124, whether they be fixed to the user (e.g., the smart device 120) or fixed within an environment in which the gait assessor system 100 is implemented.

To further illustrate the operations of the gait assessor 134, reference is now made to an illustrative example where a user uploads a user registration including the user's name, type of smart device 120 type, and type of inertial sensors 124, along with a link to the user's medical records.

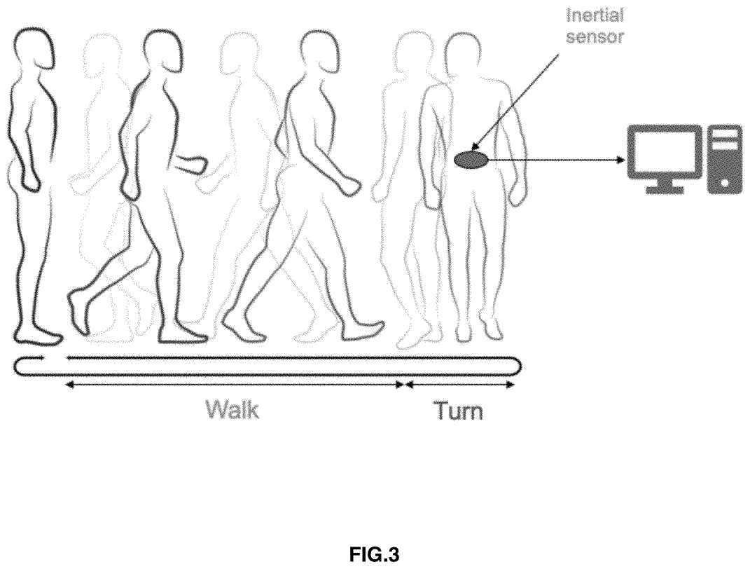

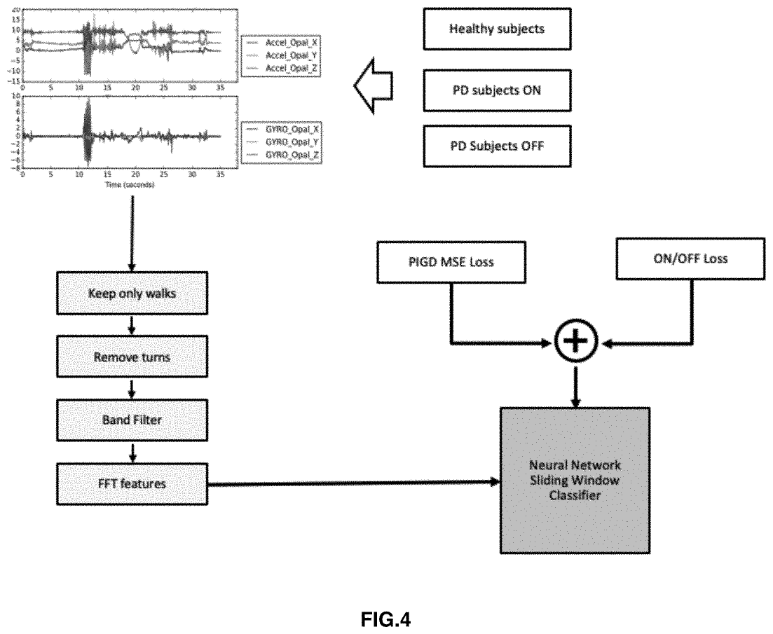

The gait assessor 134 may receive and process training data (step 206). In embodiments, the gait assessor 134 may receive training data via user upload, from databases, or from the inertial sensors 124. With reference to FIG. 4, training data may be in any form representing an individual's walking motion, for example, a graph of acceleration over time (depicted in FIG. 4), a graph of gyration over time, tables, charts, lists, etc. In preferred embodiments, and as illustrated by FIG. 3, the training data is collected by an inertial sensor as an individual walks back and forth in a straight line and turns. The training data may be received for one or more classifications of user gait, for example baseline training data may be received for healthy individuals who have not been diagnosed with a disease (healthy subjects), individuals who have been diagnosed with a disease but have mild symptoms due to taking medication (PD subjects ON), and individuals who have been diagnosed with a disease and who exhibit worsening symptoms, for example due to their medication wearing off (PD subjects OFF). In embodiments, training data for additional or different classifications of individuals may be collected, for example, individuals who have been diagnosed with a disease and exhibit moderate symptoms. Upon receiving training data, the gait assessor 134 may process the collected raw training data. In embodiments, the gait assessor 134 may process the collected training data via amplification, filtering, and performing other data refining operations on the data, such as the Fast Fourier Transform (FFT). In a preferred embodiment, the gait assessor 134 may only process data collected by the inertial sensor 124 as an individual is walking in a straight line, and disregard/discard data collected by the inertial sensor 124 as an individual is turning around. The gait assessor 134 may determine that the individual is turning around based on the inertial sensors 124, for example a lack of velocity, decreased acceleration, an increase in gyroscopic motion, etc. The gait assessor 134 may further apply a band filter to the training data to remove high frequency noise and the effect of gravity. The data may further be processed by applying the Fast Fourier Transform (FFT) to each time series and keeping the resulting FFT amplitudes as features. In general, the gait assessor 134 may implement any data refining and processing operations in order to prepare the data for gait assessment.

With reference again to the previously introduced example where the user uploads a user registration, the gait assessor 134 receives training data from the inertial sensor 124 of healthy individuals who have not been diagnosed with Parkinson's disease (healthy subjects), individuals who have been diagnosed with Parkinson's disease and have mild symptoms due to taking medication such as Levodopa (PD subjects ON), and individuals who have been diagnosed with Parkinson's disease and who exhibit worsening symptoms due to their medication wearing off (PD subjects OFF) as they walking back and forth across a room. The gait assessor 134 removes the training data corresponding to the individuals turning, applies a band filter to the resulting data, and then applies FFT features to process the data.

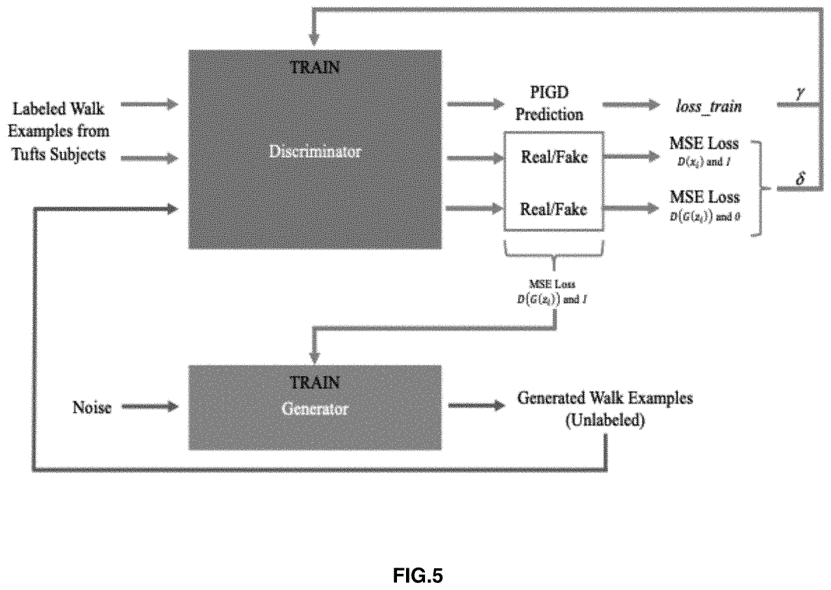

The gait assessor 134 may train one or more gait assessor models 132 based on the collected and processed training data (step 208). The gait assessor models 132 may be one or more algorithms modelling a correlation between one or more features or attributes of training data and a gait assessment of an individual, such as healthy subject, PD subjects ON, or PD subjects OFF. The one or more features may include characteristics relating to a an individual's gait, such as tremors, slowness of movement, muscular rigidity, gait speed, stride length, toe off angle, strike angle, trunk coronal range of motion, body symmetry, etc. In embodiments, the gait assessor models 132 may weight the features based on an effect that the one or more features have on the assessment of an individual's gait. The gait assessor 134 may further consider an individual's posture and gait section (PIGD) score of the unified Parkinson's disease rating scale (UPDRS) and an individual's ON/OFF Loss in the training of the gait assessor models 132. PIGD Loss may refer to how well the output of the model matches the PIGD score given to the individual by the individual's doctor or medical professional. ON/OFF Loss may refer to how well the change in PIGD score between ON and OFF states of the same individual matches the expected behavior (i.e. PIGD score is higher in OFF states and lower in ON states). In embodiments, a final loss value may be determined by calculating a weighted average of PIGD Loss and ON/OFF Loss values. For example, the gait assessor 134 may multiply the PIGD Loss and ON/OFF Loss values by predefined weights to calculate a final loss value. In embodiments, the gait assessor 134 may generate the gait assessor models 132 using machine learning methods, such as neural networks, deep learning, hierarchical learning, Gaussian Mixture modelling, Hidden Markov modelling, and K-Means, K-Medoids, or Fuzzy C-Means learning, etc. In the example embodiment, and with reference to FIG. 5, the gait assessor 134 may utilize generative adversarial nets (GAN) to train the gait assessor models 132. Using a GAN, the gait assessor 134 may utilize a generator to generate additional training data in the form of unlabeled walk examples based on the previously collected data. The gait assessor 134 may then use a discriminator to discriminate between the generated training data (fake) and the collected labeled training data (real). Based on whether the discriminator correctly discriminates between the generated and collected data, the gait assessor 134 may adjust the gait assessor models used by the discriminator in assessing gait. The gait assessor 134 utilizes the gait assessor models 132 to determine a PIGD prediction and Real/Fake assessment, which provide feedback to the generator and discriminator gait assessor models 132. The gait assessor 134 uses this feedback to more accurately train the gait assessor models 132 with each additional iteration through the GAN training method.

With reference again to the previously introduced example where the gait assessor 134 receives and processes training data, the gait assessor 134 considers the individuals' PIGD score and ON/OFF Loss, and utilizes a GAN training method to train the gait assessor models 132.

The gait assessor 134 may collect and process user gait data (step 210). In embodiments, the gait assessor 134 may collect user gait data via user input, from databases, or from the inertial sensors 124. With reference to FIG. 4, user gait data may be in any form representing a user's walking motion, for example, a graph of acceleration over time (depicted in FIG. 4), a graph of gyration over time, tables, charts, lists, etc. In preferred embodiments, and as illustrated by FIG. 3, the user gait data is collected by an inertial sensor as the user walks back and forth in a straight line and turns. Upon receiving user gait data, the gait assessor 134 may process the collected raw user gait data. In embodiments, the gait assessor 134 may process the collected user gait data via amplification, filtering, and performing other data refining operations on the data, such as the Fast Fourier Transform (FFT). In a preferred embodiment, the gait assessor 134 may only process data collected by the inertial sensor 124 as the user is walking in a straight line, and disregard/discard data collected by the inertial sensor 124 as the user is turning around. The gait assessor 134 may determine that the user is turning around based on the inertial sensors 124, for example a lack of velocity, decreased acceleration, an increase in gyroscopic motion, etc. The gait assessor 134 may further apply a band filter to the user gait data to remove high frequency noise and the effect of gravity. The data may further be processed by applying the Fast Fourier Transform (FFT) to each time series and keeping the resulting FFT amplitudes as features. In general, the gait assessor 134 may implement any data refining and processing operations in order to prepare the data for gait assessment.

With reference again to the previously introduced example where the gait assessor 134 utilizes a GAN training method to more accurately train the gait assessor models 132, the gait assessor 134 collects user gait data from the inertial sensors 124, removes the data corresponding to the user turning, applies a band filter to the resulting data, and then applies FFT features to process the data.

The gait assessor 134 may apply one or more gait assessor models 132 to assess a user's gait (step 212). In embodiments, the gait assessor models 132 may be applied to one or more extracted features to compute an assessment of a user's gait, such as high Parkinson's effects, low Parkinson's effects, and no Parkinson's effects. As previously mentioned, such extracted features may include tremors, slowness of movement, muscular rigidity, gait speed, stride length, toe off angle, strike angle, trunk coronal range of motion, body symmetry, etc., and the one or more gait assessment models 132 may be generated through machine learning techniques such as neural networks. In these embodiments, the gait assessor 134 may weight the extracted features such that features shown to have a greater correlation with a correct assessment of gait are weighted greater than those features that are not. In some embodiments, the gait assessor 134 may treat the user's gait data and gait assessment as training data for the gait assessment of a future user. In this way, the gait assessor 134 may modify and/or update its gait models 132.

With reference again to the previously introduced example where the gait assessor 134 collects user gait data from the inertial sensors 124 and processes the collected data, the gait assessor 134 assesses the user as having a gait of a healthy subject, and uses the user's gait data and user's gait assessment as training data to update the gait models 132.

Upon the gait assessor 134's assessment of a user's gait, the gait assessor 134 may convey the user's gait assessment to the user (step 214). The gait assessor 134 may convey the user's gait assessment to the user in the form of audio, video, text, or any other manner. In embodiments, the gait assessor 134 may convey the user's gait assessment to the user by updating the user's health records or notifying the user's physician of the user's gait assessment.

With reference again to the previously introduced example where the gait assessor 134 assesses the user as having a gait of a healthy subject, the gait assessor 134 notifies the user and the user's physician of the user's healthy subject gait assessment.

FIG. 3 depicts an exemplary schematic diagram of an inertial sensor 124 of the gait assessor system 100 positioned on a user's lower back. In embodiments, a user wearing the inertial sensor 124 may walk forward from a starting point in a straight line, stop and turn around 180 degrees, and walk forward in a straight line until returning to the starting point. In embodiments, a user may repeat these actions any number of times to collect user gait data.

FIG. 4-5 depict exemplary flowcharts illustrating the operations of the gait assessor 134 of the gait assessor system 100 in training the gait assessor models 132 based on training data.

The gait assessor models 132 may be a neural network sliding window classifier modelling a correlation between one or more features or attributes of training data and a gait assessment of a an individual, such as healthy subject, PD subjects ON, or PD subjects OFF. The one or more features may include characteristics relating to a an individual's gait, such as tremors, slowness of movement, muscular rigidity, gait speed, stride length, toe off angle, strike angle, trunk coronal range of motion, body symmetry, etc. In embodiments, the gait assessor models 132 may weight the features based on an effect that the one or more features have on the assessment of a individual's gait. The gait assessor 134 may further consider an individual's posture and gait section (PIGD) score of the unified Parkinson's disease rating scale (UPDRS) and an individual's ON/OFF Loss in the training of the gait assessor models 132. PIGD Loss may refer to how well the output of the model matches the PIGD score given to the individual by the individual's doctor or medical professional. ON/OFF Loss may refer to how well the change in PIGD score between ON and OFF states of the same individual matches the expected behavior (i.e. PIGD score is higher in OFF states and lower in ON states). In embodiments, a final loss value may be determined by calculating a weighted average of PIGD Loss and ON/OFF Loss values. For example, the gait assessor 134 may multiply the PIGD Loss and ON/OFF Loss values by predefined weights to calculate a final loss value. In embodiments, the gait assessor 134 may generate the gait assessor models 132 using machine learning methods, such as neural networks, deep learning, hierarchical learning, Gaussian Mixture modelling, Hidden Markov modelling, and K-Means, K-Medoids, or Fuzzy C-Means learning, etc. In the example embodiment, and with reference to FIG. 5, the gait assessor 134 may utilize generative adversarial nets (GAN) to train the gait assessor models 132. Using a GAN, the gait assessor 134 may utilize a generator to generate additional training data in the form of unlabeled walk examples based on the previously collected data. The gait assessor 134 may then use a discriminator to discriminate between the generated training data (fake) and the collected labeled training data (real). Based on whether the discriminator correctly discriminates between the generated and collected data, the gait assessor 134 may adjust the gait assessor models used by the discriminator in assessing gait. The gait assessor 134 utilizes the gait assessor models 132 to determine a PIGD prediction and Real/Fake assessment, which provide feedback to the generator and discriminator gait assessor models 132. The gait assessor 134 uses this feedback to more accurately train the gait assessor models 132 with each additional iteration through the GAN training method.

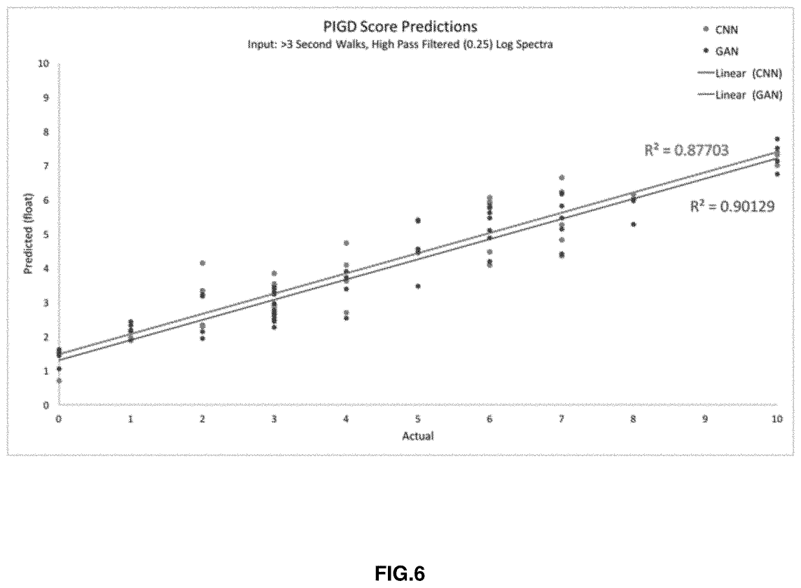

FIG. 6 depicts a graph of results of the gait assessor 134 trained according to the method depicted in FIG. 4-5. The confidence value of PIGD score predictions using the GAN method of FIG. 4-5 is 0.90129, which is higher than the confidence value of PIGD score predictions using Convolutional Neural Networks (CNN), which is 0.87703.

FIG. 7 depicts a block diagram of devices within the gait assessor 134 of the gait assessor system 100 of FIG. 1, in accordance with the exemplary embodiments. It should be appreciated that FIG. 7 provides only an illustration of one implementation and does not imply any limitations with regard to the environments in which different embodiments may be implemented. Many modifications to the depicted environment may be made.

Devices used herein may include one or more processors 02, one or more computer-readable RAMs 04, one or more computer-readable ROMs 06, one or more computer readable storage media 08, device drivers 12, read/write drive or interface 14, network adapter or interface 16, all interconnected over a communications fabric 18. Communications fabric 18 may be implemented with any architecture designed for passing data and/or control information between processors (such as microprocessors, communications and network processors, etc.), system memory, peripheral devices, and any other hardware components within a system.

One or more operating systems 10, and one or more application programs 11 are stored on one or more of the computer readable storage media 08 for execution by one or more of the processors 02 via one or more of the respective RAMs 04 (which typically include cache memory). In the illustrated embodiment, each of the computer readable storage media 08 may be a magnetic disk storage device of an internal hard drive, CD-ROM, DVD, memory stick, magnetic tape, magnetic disk, optical disk, a semiconductor storage device such as RAM, ROM, EPROM, flash memory or any other computer-readable tangible storage device that can store a computer program and digital information.

Devices used herein may also include a R/W drive or interface 14 to read from and write to one or more portable computer readable storage media 26. Application programs 11 on said devices may be stored on one or more of the portable computer readable storage media 26, read via the respective R/W drive or interface 14 and loaded into the respective computer readable storage media 08.

Devices used herein may also include a network adapter or interface 16, such as a TCP/IP adapter card or wireless communication adapter (such as a 4G wireless communication adapter using OFDMA technology). Application programs 11 on said computing devices may be downloaded to the computing device from an external computer or external storage device via a network (for example, the Internet, a local area network or other wide area network or wireless network) and network adapter or interface 16. From the network adapter or interface 16, the programs may be loaded onto computer readable storage media 08. The network may comprise copper wires, optical fibers, wireless transmission, routers, firewalls, switches, gateway computers and/or edge servers.

Devices used herein may also include a display screen 20, a keyboard or keypad 22, and a computer mouse or touchpad 24. Device drivers 12 interface to display screen 20 for imaging, to keyboard or keypad 22, to computer mouse or touchpad 24, and/or to display screen 20 for pressure sensing of alphanumeric character entry and user selections. The device drivers 12, R/W drive or interface 14 and network adapter or interface 16 may comprise hardware and software (stored on computer readable storage media 08 and/or ROM 06).

The programs described herein are identified based upon the application for which they are implemented in a specific one of the exemplary embodiments. However, it should be appreciated that any particular program nomenclature herein is used merely for convenience, and thus the exemplary embodiments should not be limited to use solely in any specific application identified and/or implied by such nomenclature.

Based on the foregoing, a computer system, method, and computer program product have been disclosed. However, numerous modifications and substitutions can be made without deviating from the scope of the exemplary embodiments. Therefore, the exemplary embodiments have been disclosed by way of example and not limitation.

It is to be understood that although this disclosure includes a detailed description on cloud computing, implementation of the teachings recited herein are not limited to a cloud computing environment. Rather, the exemplary embodiments are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or data center).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

Referring now to FIG. 8, illustrative cloud computing environment 50 is depicted. As shown, cloud computing environment 50 includes one or more cloud computing nodes 40 with which local computing devices used by cloud consumers, such as, for example, personal digital assistant (PDA) or cellular telephone 54A, desktop computer 54B, laptop computer 54C, and/or automobile computer system 54N may communicate. Nodes 40 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 8 are intended to be illustrative only and that computing nodes 40 and cloud computing environment 50 can communicate with any type of computerized device over any type of network and/or network addressable connection (e.g., using a web browser).

Referring now to FIG. 9, a set of functional abstraction layers provided by cloud computing environment 50 (FIG. 8) is shown. It should be understood in advance that the components, layers, and functions shown in FIG. 9 are intended to be illustrative only and the exemplary embodiments are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and gait assessment 96.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A computer-implemented method for assessing gait, the method comprising:
collecting data corresponding to a walking user; and
assessing a gait of the user based on applying one or more models to the data.

2. The method of claim 1, further comprising:
receiving and processing training data; and
training one or more models based on the training data;

3. The method of claim 1, wherein the one or more models are further trained based on one or more Postural Instability and Gait Disturbance (PIGD) scores and one or more ON/OFF Loss values, wherein the one or more ON/OFF Loss values are indicative of whether the PIGD score of an individual in the OFF state is higher than the PIGD score of the individual in the ON state.

4. The method of claim 1, wherein the one or more models are trained using generative adversarial nets.

5. The method of claim 1, wherein the one or more models are trained by:
extracting one or more features from the training data; and
training one or more models based on the one or more features.

6. The method of claim 1, further comprising:
extracting one or more features from the user's gait data; and
assessing the user's gait based on applying the one or more models to the extracted one or more features.

7. The method of claim 6, wherein the one or more features include features selected from a group comprising tremors, slowness of movement, muscular rigidity, gait speed, stride length, toe off angle, strike angle, trunk coronal range of motion, and body symmetry.

8. A computer program product for assessing gait, the computer program product comprising:
one or more non-transitory computer-readable storage media and program instructions stored on the one or more non-transitory computer-readable storage media capable of performing a method, the method comprising:
collecting data corresponding to a walking user; and
assessing a gait of the user based on applying one or more models to the data.

9. The computer program product of claim 8, further comprising:
receiving and processing training data; and
training one or more models based on the training data;

10. The computer program product of claim 8, wherein the one or more models are further trained based on one or more Postural Instability and Gait Disturbance (PIGD) scores and one or more ON/OFF Loss values, wherein the one or more ON/OFF Loss values are indicative of whether the PIGD score of an individual in the OFF state is higher than the PIGD score of the individual in the ON state.

11. The computer program product of claim 8, wherein the one or more models are trained using generative adversarial nets.

12. The computer program product of claim 8, wherein the one or more models are trained by:
extracting one or more features from the training data; and
training one or more models based on the one or more features.

13. The computer program product of claim 8, further comprising:
extracting one or more features from the user's gait data; and
assessing the user's gait based on applying the one or more models to the extracted one or more features.

14. The computer program product of claim 13, wherein the one or more features include features selected from a group comprising tremors, slowness of movement, muscular rigidity, gait speed, stride length, toe off angle, strike angle, trunk coronal range of motion, and body symmetry.

15. A computer system for assessing gait, the computer system comprising:
one or more computer processors, one or more computer-readable storage media, and program instructions stored on the one or more of the computer-readable storage media for execution by at least one of the one or more processors capable of performing a method, the method comprising:
collecting data corresponding to a walking user; and
assessing a gait of the user based on applying one or more models to the data.

16. The computer system of claim 15, further comprising:
receiving and processing training data; and
training one or more models based on the training data;

17. The computer system of claim 15, wherein the one or more models are further trained based on one or more Postural Instability and Gait Disturbance (PIGD) scores and one or more ON/OFF Loss values, wherein the one or more ON/OFF Loss values are indicative of whether the PIGD score of an individual in the OFF state is higher than the PIGD score of the individual in the ON state.

18. The computer system of claim 15, wherein the one or more models are trained using generative adversarial nets.

19. The computer system of claim 15, wherein the one or more models are trained by:
extracting one or more features from the training data; and
training one or more models based on the one or more features.

20. The computer system of claim 15, further comprising:
extracting one or more features from the user's gait data; and
assessing the user's gait based on applying the one or more models to the extracted one or more features.
